(19) United States
(12) Patent Application Publication
(10) Pub. No.: US 2016/0051417 A1
(43) Pub. Date: Feb. 25, 2016

(54) Urine Detection Inductor Suitable For Large-scale Production

(71) Applicant: Joseph CHIU, Sheung Wan, Hong Kong (CN)

(72) Inventor: Joseph CHIU, Hong Kong (CN)

(21) Appl. No.: 14/781,315

(22) PCT Filed: Nov. 6, 2014
(86) PCT No.: PCT/IB2014/065846

§ 371 Date: Sep. 30, 2015

(30) Foreign Application Priority Data
Nov. 6, 2013 (HK) 13112461.0
Nov. 25, 2013 (CN) 201310605588.9
Nov. 25, 2013 (CN) 201320745026.X

(51) Int. Cl. A61F 13/42; A61L 15/18; A61L 15/56

(52) U.S. Cl. CPC A61F 13/42; A61L 15/56; A61L 15/18

(57) ABSTRACT

A detector for wet diaper is suitable for large-scale production. The detector comprises a nonwoven fabric (1) and at least two electrodes (2, 3) which are carbon powder coated on the nonwoven fabric (1). The length of the electrodes (2, 3) is 2-20 mm while the length thereof is more than 3 times than that of the width. The carbon powder is adaptable to the skin of the infants, the old, and the patients. It will not cause allergy or oppression to the skin. Further, the carbon layer possesses the function of filtration and odor adsorption, which is conducive to the hygiene and health of the subjects, such as, the infant, the old and the patients.

### CROSS-REFERENCE TO RELATED APPLICATION

This application claims benefit of Hong Kong Short-term Patent having Patent No. HK1191503 filed on 6 Nov. 2013; Chinese Invention Application having Application No. 201310605588.9 filed on 25 Nov. 2013; and Chinese Utility Model Patent having Patent No. ZL201320745026.X, which are hereby incorporated by reference herein in its entirety.

### FIELD OF INVENTION

This invention relates to a detecting device, especially a detector for wet diaper that is suitable for large-scale production.

### BACKGROUND OF INVENTION

Babies frequently wet their diapers and wear the wet diapers for prolonged periods before they are noticed and changed since it is not always easy to notice such wet condition promptly, especially for young parents who lack experience and are too busy. Such wet condition in a diaper not only causes discomfort to babies but also leads to the problem known as “diaper rash”, which seriously threatens the health of babies. The same issue will also be present when taking care the old and patients suffering from hemiplegia, incontinence, etc.

There are many kinds of diapers with moisture detector and alarm in the market. They comprise the detecting part, external circuit and alarm device. The detecting part is the core thereof.

Generally, the detecting part comprises snap-fastened electrode or un-attached flexible conductive medium affixed to a diaper. The electrodes on the diaper are attached to the switching circuit through flexible wire. The export end of the switching circuit is attached to an alarm device and a power source. When the diaper is wet by urine, part of the flexible conductive medium is connected as a result, which will initiate the detecting part and alarm device to remind the parents or caretakers by triggering music, etc. so as to protect the babies from the irritation and harm caused by urine.

Normally, the flexible conductive medium is made of humidity-dependent sensor, conductive rubber or conductive plastic, fine metal, etc., which cannot be well integrated with the material of a diaper. Their being close to the body and the resulted oppression will cause discomfort, especially to the delicate skin of the infants. Further, the conductivity is not very good when there is urine.

### SUMMARY OF INVENTION

In the light of the foregoing background, it is an object of the present invention to provide a detector for wet diaper, which is more efficient and safer to use as well as suitable for large-scale production.

Accordingly, the present invention, in one aspect, provides an apparatus for detecting wet condition in a diaper, comprising detector, said detector comprises nonwoven fabric being sized to said diaper, a first electrode and a second electrode parallel to each other residing on nonwoven fabric and insulated with each other, and a third electrode residing on nonwoven fabric insulated with the first and second electrode, wherein the first, second, and third electrodes are carbon powder coated on the nonwoven fabric; the resistance between any two electrodes decreases as a result of a wetted diaper being indicative of said wet condition.

In an exemplary embodiment of the present invention, the third electrode is between the first electrode and the second electrode; the distance between the third electrode and the first electrode or the second electrode is smaller than that between the first electrode and the second electrode.

In another exemplary embodiment of the present invention, the third electrode is vertical to the first electrode and the second electrode; the distance between the third electrode and the first electrode or the second electrode is greater than that between the first electrode and the second electrode.

In another exemplary embodiment of the present invention, the width of the first electrode or second electrode is 2-20 mm while the length of the first electrode or the second electrode is more than 3 times greater than the width thereof.

In another exemplary embodiment of the present invention, the first electrode and the second electrode are strip in shape.

In an exemplary embodiment of the present invention, a first layer of nonwoven fabric is set above said detector; the density of the first layer of nonwoven fabric is lower than that of the nonwoven fabric which the electrodes reside in.

In a further aspect of the present invention, an apparatus for detecting wet condition in a diaper suitable for large-scale production is provided, comprising detector, the detector comprises nonwoven fabric being sized to be attached to a surface of said diaper, and at least a first electrode and a second electrode parallel to each other residing in the nonwoven fabric and insulated with each other, wherein the first and second electrodes are carbon powder coated on the nonwoven fabric.

In one exemplary embodiment, the detector further comprises a third electrode and a fourth electrode; the third and fourth electrodes are between the first and second electrodes. The above electrodes are carbon powder coated on the nonwoven fabric and are electrically insulated with each other.

In another exemplary embodiment, the first and second electrodes are strip in shape and parallel to each other.

In another exemplary embodiment, the width of the first electrode or second electrode is 2-20 mm while the length of the first electrode or second electrode is more than 3 times greater than the width thereof.

In another exemplary embodiment, the third electrode is vertical to the first and second electrodes.

In one embodiment, the third electrode serves as a switch in the circuit.

In another exemplary embodiment, the first electrode and the second electrode are coated on the nonwoven fabric as concentric circles.

In another exemplary embodiment, the first electrode is coated on said nonwoven fabric as a circle while the second electrode is coated on the nonwoven fabric as a circular concentric with the first electrode.

In another exemplary embodiment, a first layer of nonwoven fabric is set above the detector while the density of the first layer of nonwoven fabric is lower than that of the nonwoven fabric in which the electrodes reside.

In another exemplary embodiment, the first layer of nonwoven is attached to the nonwoven fabric in which the electrodes reside by adhesive.

In another exemplary embodiment, the resistance of interval without coated carbon powder between the third electrode and the first electrode or the second electrode is more than 10 times than the resistance of interval with coated carbon powder.

There are many advantages of the present invention. As a flexible conductive medium, for example, the carbon powder has nice adaptability to the skin of the infant, the old and the patients without causing oppression or allergy, etc. Meanwhile, the carbon layer possesses the features of filtration and odor adsorption, which is conducive to the 100 hygiene and health of the users.

The nonwoven fabric used in the present invention is soft, resilient, durable, flame-retardant, antistatic, non-poisonous, non-irritant, of no peculiar smell, fiber tow free, can be molded by one compression. The nonwoven fabric is manufactured by FDA food-grade material without any additional chemical component. It has the feeling of 105 cotton while it is easy to be shaped and less expensive.

Other advantages of the invention include the portable size of the detector, higher sensitivity, and the relatively simple manufacturing process thereof.

### BRIEF DESCRIPTION OF FIGURES

FIG. 1 is a plan view of the detector for wet diaper with electrodes in strip shape.

FIG. 2 is a plan view of the detector for wet diaper with electrodes arranged as concentric circles.

FIG. 3 is a plan view of the detector for wet diaper with electrodes arranged as concentric circle and concentric circular.

FIG. 4 is a plan view of the detector for wet diaper with four electrodes coated on the nonwoven fabric.

FIG. 5 is a sectional view of the circuit arrangement, wherein 1 refers to nonwoven fabric; 5 refers to the first layer of the nonwoven fabric; and 6 refers to the adhesive.

FIG. 6 is the circuit arrangement of the detector for wet diaper.

### DETAILED DESCRIPTION OF THE PREFERRED EMBODIMENTS

As used herein and in the claims, “comprising” means including the following elements but not excluding others.

As used herein and in the claims, “connect” refers to electrical connection either directly or indirectly via one or more electrical means unless otherwise stated.

It is an object of the present invention to provide a detector for wet diaper, which is more efficient and safer as well as suitable or large-scale production.

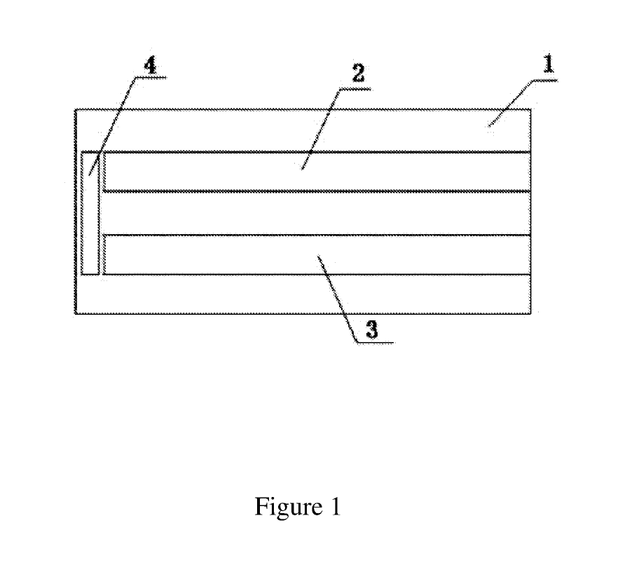

Referring to FIG. 1, a detector for wet diaper that is suitable for large-scale production is provided, comprising nonwoven fabric 1, a first electrode 2 and a second electrode 3, which are carbon powder coated on the nonwoven fabric 1 in parallel. The width of the first electrode 2 or the second electrode 3 is 2-20 mm while the length is more than 3 times than the width thereof. The first electrode 2 is insulated with second electrode 3. A third electrode 4 is set vertical to the first electrode 2 and the second electrode 3. The three electrodes are insulated with each other. The distance between the third electrode 4 and the first electrode 2 or the second electrode 3 is smaller than the distance between the first electrode 2 and the second electrode 3. Such arrangement increases the sensitivity of the urine detection since not only wetting the interval between the first electrode 2 and the second electrode 3 would trigger the alarm, wetting the intervals between either the first electrode 2 and the third electrode 4 or the second electrode 3 and the third electrode 4 would also trigger the alarm.

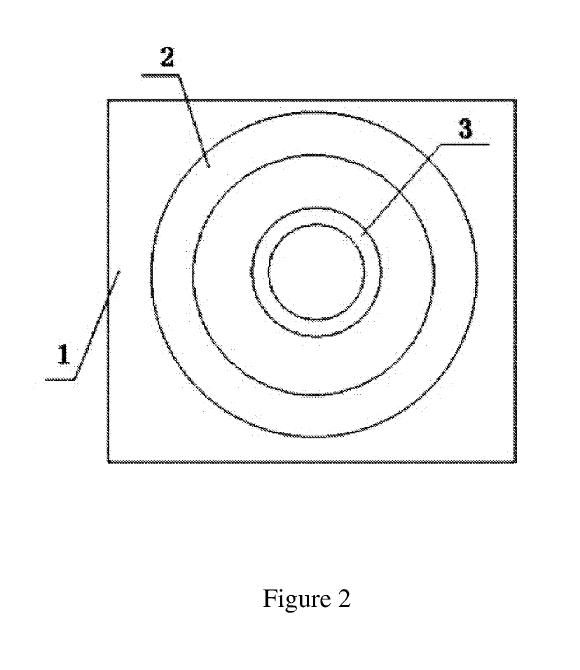

Referring to FIG. 2, a detector for wet diaper that is suitable for large-scale production is provided, comprising nonwoven fabric 1, a first electrode 2 and a second electrode 3 residing on the nonwoven fabric. The first electrode 2 and the second electrode 3 are carbon powder coated on the nonwoven fabric 1 in concentric circles. The diameter of the first electrode 2 is larger than that of the second electrode 3. The first electrode 2 and the second electrode 3 are insulated with each other.

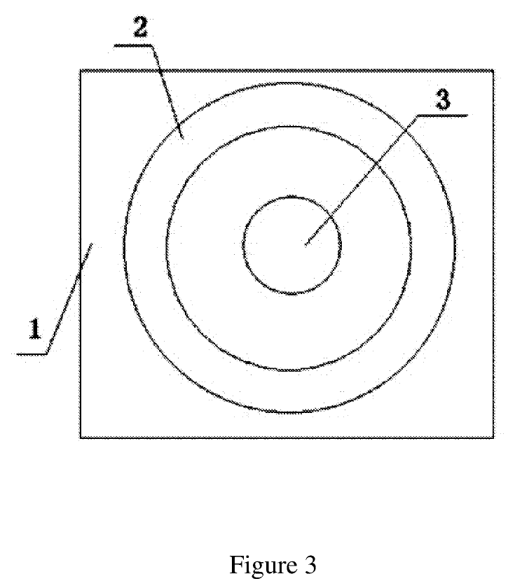

Referring to FIG. 3, a detector for wet diaper that is suitable for large-scale production is provided, comprising nonwoven fabric 1, a first electrode 2 and a second electrode 3 residing on the nonwoven fabric 1. The first electrode 2 is carbon powder coated on the nonwoven fabric in a circle while the second electrode 3 is of disc-shape that is concentric with the first electrode 2.

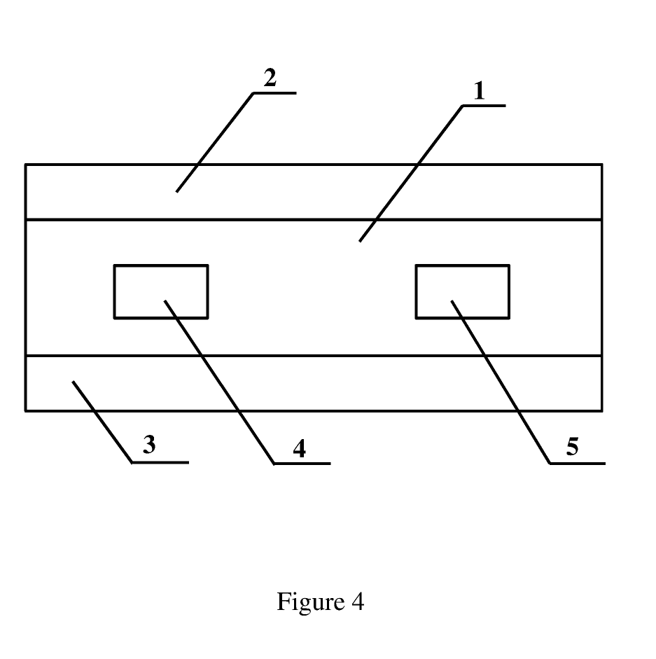

Referring to FIG. 4, a detector for wet diaper that is suitable for large-scale production is provided, comprising nonwoven fabric 1, a first electrode 2 and a second electrode 3 are coated on the nonwoven fabric 1. A third electrode 4 and a fourth electrode 5 are set between the first electrode 2 and the second electrode 3 on the nonwoven fabric 1. The above electrodes are electrically insulated with each other.

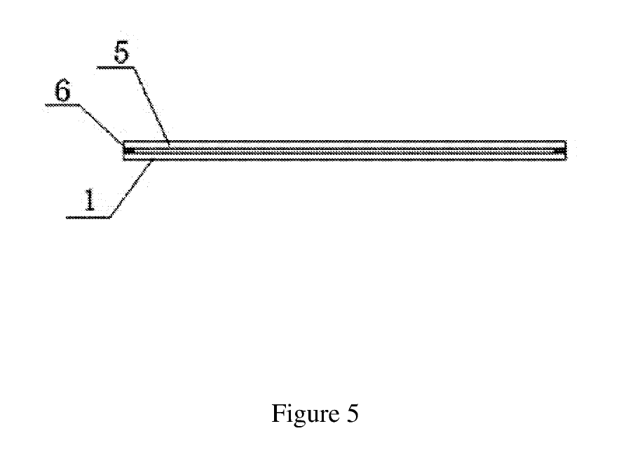

Referring to FIG. 5, a detector for wet diaper that is suitable for large-scale production, comprising nonwoven fabric 1 and a first layer of nonwoven fabric 5. The first layer of nonwoven fabric 5 is set above the detector. The density of the first layer of the nonwoven fabric 5 is lower than that of the nonwoven fabric 1 used in the detector. The first layer nonwoven fabric 5 is attached with the nonwoven fabric 1 through adhesive 6. Since the density of the first layer of nonwoven fabric 5 is lower than that of the nonwoven fabric 1 used in the detector, when there is urine on the first layer of nonwoven fabric 5, the urine can easily penetrate to the nonwoven fabric 1 used in the detector. The urine on the nonwoven fabric 1 can be detected by single chip micyoco (SCM) and thereby triggers the wet alarm.

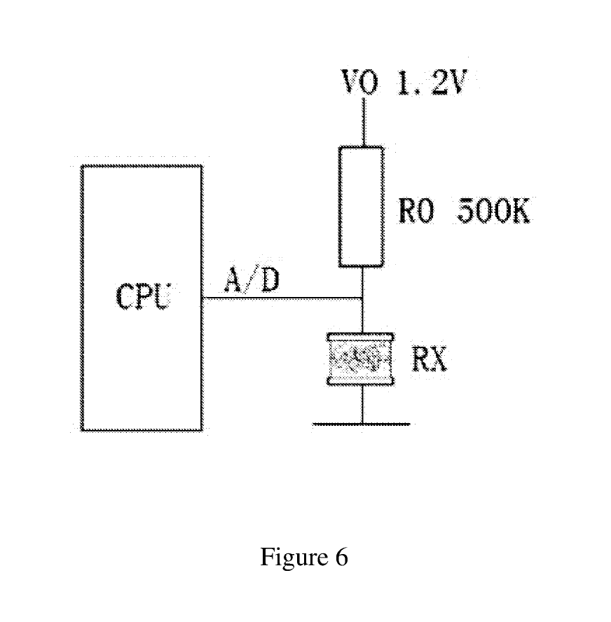

Referring to FIG. 6, the circuit for a detector for wet diaper is illustrated. Since urine is conductive, the presence of urine on the nonwoven fabric 1 leads to the decrease of resistance RX of the originally insulated flexible conductive medium of the carbon layer on the nonwoven fabric 1 from infinity to a state of low conductivity, in which RX represents the resistance between the first electrode 2 and the second electrode 3. A standard resistor RO of 500 k is connected to RX in series. The other end of RO is connected to a steady voltage of 1.2V while the other end of RX is connected to a negative terminal voltage. The electrical connector terminals of RO and RX are connected to A/D import terminal. The voltage variation is identified by a SCM to detect if there is urine on the nonwoven fabric 1 and thereby triggers the wet alarm accordingly.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the first and the second electrodes as described in FIG. 1 are strips in parallel to each other, but it is clear that other shapes may be used according to the user's need and preference, such as, circles as illustrated in FIG. 2, circulars as illustrated in FIG. 3, curves, etc., which are in parallel positions. In other words, the first electrode can be of any shape while the second electrode is in the corresponding shape with a distance from the first electrode. The two electrodes are in parallel and insulated with each other.

The number and configuration of the electrodes are not limited to the above examples. For instance, the distance between the third electrode and the first electrode or the second electrode can vary depending on users' requirement. In one embodiment, one of the electrodes serves as a switch in the circuit. Further, the number of electrodes can vary depending on users' need. For example, a third and a fourth electrodes can be printed between the first and the second electrodes on the nonwoven fabric. The above electrodes are insulated with each other.

The present invention employs nonwoven fabric. The nonwoven fabric is fabric that was not gone through weaving or knitting process. It is obtained by following the below steps: constructing a web through directly binding and interlocking polymer slice, staple fiber, or filaments mechanically or by air streams; reinforcing the resultant by hydro-entanglement, needlepunching, or calendering through heated rollers; and obtaining the nonwoven fabric by post continuous process.

What is claimed is:

1. An apparatus for detecting wet condition in a diaper comprising a detector, wherein said detector comprising
a) nonwoven fabric being sized to be attached to a surface of said diaper;
b) a first electrode and a second electrode parallel to each other residing in said nonwoven fabric and insulated with each other;
c) a third electrode located on said nonwoven fabric and insulated with said first and second electrode; and
wherein said first, second, and third electrodes are carbon powder coated on said nonwoven fabric; the resistance between any two electrodes decreases as a result of a wetted diaper being indicative of said wet condition.

2. The detector according to claim 1, wherein said third electrode is between said first electrode and said second electrode; the distance between said third electrode and said first electrode or said second electrode is smaller than that between said first electrode and said second electrode.

3. The detector according to claim 1, wherein said third electrode is vertical to said first electrode and said second electrode; the distance between said third electrode and said first electrode or said second electrode is greater than that between said first electrode and said second electrode.

4. The detector according to claim 1, wherein the width of said first electrode or second electrode is 2-20 mm; the length of said first electrode or second electrode is more than 3 times greater than the width thereof.

5. The detector according to claim 1, wherein said first electrode and said second electrode are strip in shape.

6. The detector according to claim 1, wherein a first layer of nonwoven fabric is set above said detector; the density of said first layer of nonwoven fabric is lower than that of said nonwoven fabric in which said electrodes reside.

7. An apparatus for detecting wet condition in a diaper comprising a detector, wherein said detector comprising
a) nonwoven fabric being sized to be attached to a surface of said diaper; and
b) at least a first electrode and a second electrode insulated with each other residing in said nonwoven fabric;
wherein said electrodes are carbon powder coated on said nonwoven fabric.

8. The detector according to claim 7, further comprising a third electrode and a fourth electrode, wherein said third and fourth electrodes are between said first and second electrodes.

9. The detector according to claim 7, wherein said first and second electrodes are strip in shape and parallel to each other.

10. The detector according to claim 7, wherein the width of said first electrode or second electrode is 2-20 mm; the length of said first electrode or second electrode is more than 3 times greater than the width thereof.

11. The detector according to claim 7, wherein a third electrode is vertical to said first electrode and said second electrode; said third electrode is insulated with said first electrode and said second electrode.

12. The detector according to claim 7, wherein said first electrode and said second electrode are coated on said nonwoven fabric as concentric circles.

13. The detector according to claim 7, wherein said first electrode is coated on said nonwoven fabric as a circle while said second electrode is coated on said nonwoven fabric as a circular concentric with said first electrode.

14. The detector according to claim 7, wherein a first layer of nonwoven fabric is set above said detector; the density of said first layer of nonwoven fabric is lower than the density of said nonwoven fabric in claim 7.

15. The detector according to claim 11, wherein the resistance of interval without coated carbon powder between said third electrode and said first electrode or said second electrode is more than 10 times than the resistance of interval with coated carbon powder.
